(19) United States
(12) Patent Application Publication
(10) Pub. No.: US 2022/0281794 A1
(43) Pub. Date: Sep. 8, 2022

(54) METHOD OF THE ISOLATING CHLOROSESAMONE OR CHLOROSESAMONE-CONTAINING FRACTION FROM ROOTS OF GENUS SESAMUM, AND APPLICATION OF THE FRACTION THEREOF

(71) Applicant: BRILLIANT BIOMEDICAL CORP., Keelung City (TW)

(72) Inventors: Chiou-Yueh Yeh, Taipei City (TW); Cheng-Yu Chen, Taipei City (TW); Szu-Chia Hsieh, Taipei City (TW); Po-Chieh Liao, Taipei City (TW)

(21) Appl. No.: 17/752,201

(22) Filed: May 24, 2022

Related U.S. Application Data
(63) Continuation of application No. 16/815,661, filed on Mar. 11, 2020.

(30) Foreign Application Priority Data
Mar. 15, 2019 (TW) 108108952

(51) Int. Cl. C07C 46/10; A61P 35/00

(52) U.S. Cl. CPC C07C 46/10; A61P 35/00; C07B 2200/13

(57) ABSTRACT

This invention discovers a purification method for isolating an extract containing chlorosesamone from roots of genus Sesamum plants, and its application. In this invention, D2 fraction containing chlorosesamone is obtained through an extraction step and a separation step. Then, through further purification step, chlorosesamone is obtained. In the experiment of this invention, D2 fraction or chlorosesamone extracted from roots of Sesamum indicum, a plant of genus Sesamum, can be used for the anticancer treatment for cancer cells of breast, liver, blood and lung.

### RELATED APPLICATION

This application is a continuation of U.S. patent application Ser. No. 16/815,661 filed on Mar. 11, 2022 and titled “Method of the Isolating Chlorosesamone Or Chlorosesamone-Containing Fraction from Roots of Genus Sesamum, and Application of the Fraction Thereof” which claims priority to and benefit of the filing date of Taiwan Patent Application No. 108108952 filed on Mar. 15, 2019, the entireties of which are hereby incorporated entirely herein by reference.

### FIELD OF INVENTION

The present invention relates to an isolation method of chlorosesamone or chlorosesamone-containing fraction from sesame plants, and to the composition of chlorosesamone-containing fraction , and to an application of the fraction.

### BACKGROUND OF INVENTION

Description of the Related Art

According to the top ten causes of death announced by the Ministry of Health and Welfare of Taiwan in 2017, the most common cause of death is malignant tumor and results in 48,037 deaths which is approximately equal to 28.0% of total deaths, indicating a high percentage. Furthermore, the top five cancers are lung cancer, liver cancer, colorectal cancer, breast cancer, and oral cancer, forming a threat to people in Taiwan. Therefore, cancers therapy remains highly important.

Currently, the clinical treatments of cancer mainly include immunotherapy, monoclonal antibody therapy, targeted therapy, radiation therapy, cancer surgery, and chemotherapy. However, an early detection of cancer is not easy, and tumor metastasis usually has occurred when patients noticed discomfort. In addition, the applicability of lumpectomy is limited, and most anticancer drugs not only affect the growth of normal cell, but also have strong sides effects including vomiting, limb weakness, hair loss, decreased white blood cells, and anemia, which seriously affect the patient's life quality and psychological status. Thus, the efficacy of anticancer drugs is still poor.

In recent years, the researches of Chinese herbal medicine indicate that many natural foods have anticancer effects, and thus the extraction of Chinese medicine ingredients with anticancer potentials is also an important direction for cancer therapy.

Seeds of the Pedaliaceae family are oil-rich seeds and edible, so that the sesame plants are planted globally. Among these, seeds of Sesamum indicum is one of the representative plants. Based on documents of traditional Chinese medicine, sesame seeds have benefits of body strength, liver and kidney function, internal organs nourishing, eyesight improvement, etc. In addition, the Chinese medicine has shown that sesamin purified from sesame seeds has anticancer effect on human liver and breast cancer cell. Moreover, myristic acid contained in the sesame seeds also has anticancer property. However, there is still no related researches to investigate other non-edible parts of the sesame plants for medical effect.

### SUMMARY OF THE INVENTION

The present invention discloses an isolation procedure of the Chlorosesamone or Chlorosesamone-containing fraction from roots of Sesainum indicum. In an embodiment, roots of sesame plants belonged to genus Sesamum is used for experiment, and Chlorosesamone or Chlorosesamone-containing fraction, D2 fraction are extracted from sesame roots and further apply to anticancer treatment in breast , liver , blood , and lung cancer cells.

In accordance with the present invention, an isolation method of chlorosesamone-containing fraction from roots of sesame plants includes an extraction step and a separation step. In the extraction step, roots of sesame plants are extracted at room temperature to obtain a crude extract. In the separation step, a separation is performed to obtain fractions of D1 and D2 after an ethyl acetate layer is removed from the crude extract of extraction step, wherein D2 fraction contains chlorosesamone.

By the aforementioned method, D2 fraction can be obtained and exhibits a growth inhibition effect to cancer cells.

Further, the extraction step is performed by using alcohol, n-hexane, ethyl acetate, acetone, organic solvent, or a supercritical extraction method, wherein the extraction is carried out by using roots of genus Sesamum plants plus an organic solvent with a weight ratio of 1:2 at room temperature. For example, in one embodiment, the crude extract may be formed by directly applying ethyl acetate to untreated roots. Further, the separation step uses an adsorbent resin, such as a Diaion HP20 column, which may be eluted with H2O/MeOH (weight ratio 40/60); 100% MeOH; 100% acetone; H2O/acetone (weight ratio 30/70) for the separation.

Additionally, the isolation method of the present invention to obtain chlorosesamone from roots of genus Sesamum plants includes an extraction step, a separation step and a purification step. The extraction step and the separation step are the same as those described above, so that they will not be respected. After previous two steps completed, the purification step is carried out. In the purification manipulation, D2 fraction is further purified by a silica gel column (n-hexane/benzene) at room temperature.

Further, the solvent n-hexane/ethyl acetate with a ratio of 1:1 is used for the recrystallization in a water bath with 60 degrees Centigrade to obtain chlorosesamone.

Further, chlorosesamone after the crystallization is remelted and/or dissolved by 100% dimethyl sulfoxide (DMSO), and store at −20 degrees Centigrade.

The present invention further provides a medicine application for cancer treatment and the medicine contains chlorosesamone or chlorosesamone-containing fraction , which is extracted from roots of a sesame plants. In addition, the medicine form includes powder, tablet, liquid, plaster, drink or spray.

In the data provided by this embodiment, D2 fraction has a significant effect on inhibiting growth of breast, liver, blood and lung cancer cell. In an embodiment, the 50% inhibition concentration of D2 fraction ranges from 38.2 to 181.9 μg/ml, indicating that D2 fraction has a good anticancer activity. In a preferred embodiment, the treatment time in cells is 24 hours.

### BRIEF DESCRIPTION OF THE DRAWINGS

FIG. 1 shows the structure of chlorosesamone;

FIG. 2(A) is a chromatogram of chlorosesamone processed by a high performance liquid chromatography,;

FIG. 2(B) is a chromatogram of D2 fraction processed by a high performance liquid chromatography.

FIG. 3(A) is a graph showing an experimental result of D2 fraction extracted from sesame roots. The growth inhibition of breast cancer cell in accordance with an embodiment of the present invention;

FIG. 3(B) is a graph showing an experimental result of D2 fraction extracted from sesame roots. The growth inhibition of liver cancer cell in accordance with an embodiment of the present invention;

FIG. 3(C) is a graph showing an experimental result of D2 fraction extracted from sesame roots. The growth inhibition of blood cancer cell in accordance with an embodiment of the present invention;

FIG. 3(D) is a graph showing an experimental result of D2 fraction extracted from sesame roots. The growth inhibition of lung cancer cell in accordance with an embodiment of the present invention;

FIG. 4(A) is a graph showing an experimental result of chlorosesamone that inhibits the growth of 4T1 mouse breast cancer cell in accordance with an embodiment of the present invention;

FIG. 4(B) is a graph showing an experimental result of chlorosesamone that inhibits the growth of HepG2 human liver cancer cell in accordance with an embodiment of the present invention;

FIG. 5 is a flow chart representing the isolation method of chlorosesamone-containing fraction in accordance with an embodiment of the present invention; and

FIG. 6 is a flow chart representing the purification method of chlorosesamone in accordance with an embodiment of the present invention.

### DESCRIPTION OF THE PREFERRED EMBODIMENTS

To make it easier for our examiner to understand the objective of the invention, its structure, innovative features, and performance, we use a preferred embodiment together with the attached drawings for the detailed description of the invention.

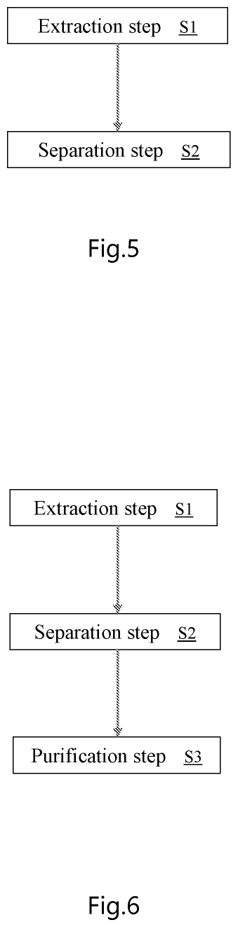

Roots of sesame plants in family Pedaliaceae is used in an embodiment for the illustration of this invention, wherein roots are washed and then extracted, wherein the whole plant, a slice or a powder of the plant is used. In FIG. 5, the isolation method of the invention includes an extraction step S1 and a separation step S2. In the extraction step S1, roots of the sesame plants are extracted at room temperature to obtain a crude extract. In the separation step S2, an ethyl acetate layer in the crude extract is obtained, and then separated to obtain fractions of D1 and D2, wherein D2 fraction contains chlorosesamone. By this procedure, D2 fraction, a composition containing Chlorosesamone, is obtained and it can effectively inhibit the growth of cancer cells.

At room temperature, an organic solvent such as alcohol, n-hexane, ethyl acetate or acetone, or a supercritical extraction method is used to obtain a crude extract, wherein roots of genus Sesamum and the organic solvent with a weight ratio of 1:2 are used for the extraction. For example, in one embodiment, the crude extract may be formed by directly applying ethyl acetate to untreated roots. After the ethyl acetate layer is obtained, an adsorbent resin, such as a Diaion HP20 column, which may be eluted with H2O/MeOH (weight ratio 40/60); 100% MeOH; 100% acetone; H2O acetone (weight ratio 30/70)), may be used for the separation to obtain fractions of D1 and D2.

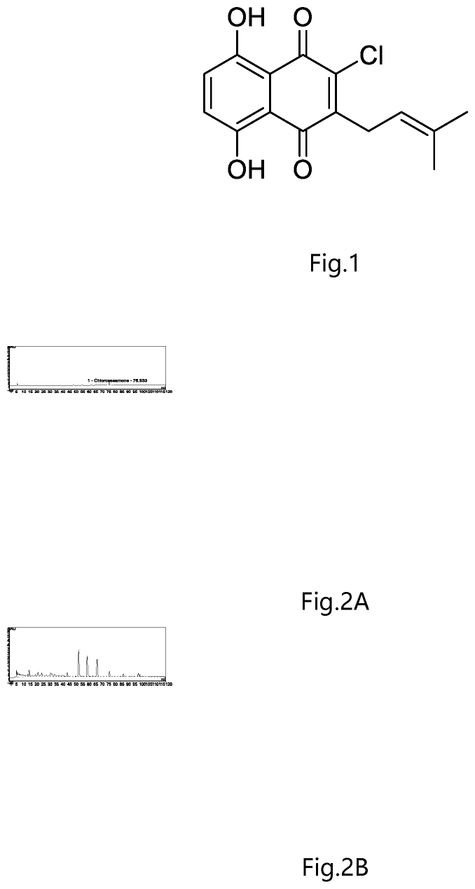

With reference to FIG. 6 for the purification method of chlorosesamone in accordance with another embodiment of the present invention, the purification method include an extraction step S1, a separation step S2 and a purification step S3. The extraction step S1 and the separation step S2 are the same as those described above, so that they will not be respected. After the steps Si and S2, the purification step S3 is performed. In the purification step S3, D2 fraction is separated by silica gel (n-hexane/benzene) and then recrystallized by the solvents n-hexane/ethyl acetate (with a ratio of 1:1) in a water bath with 60 degrees Centigrade to obtain chlorosesamone (confirmed by the NMR spectroscopy), wherein the chemical formula is shown in FIG. 1. In the analysis of the high performance liquid chromatography (HPLC), D2 fraction contains chlorosesamone, a pure compound, as shown in FIG. 2. In addition, chlorosesamone purification step also can use other related analytical instruments such as the HPLC, or other gels (such as C18; Sephadex LH2O), or a silica gel to change the mobile phase (or change the organic solvent), or a ready-made TLC plate to complete the separation operation).

In addition, the liquid containing D2 fraction or chlorosesamone is concentrated, dried, recrystallized, and then remelted and/or dissolved by 100% DMSO, and finally store at −20 degrees Centigrade.

Further, D2 fraction or chlorosesamone can be combined with other compositions to form a powder, a tablet, a liquid, a plaster, a drink or a spray, or mixed with other compositions to be made as a medicine for consuming, injection, coating, or taking. In addition, the Pedaliaceace plants can include the genus Sesamum and the genus Trapella.

The following experiments further describe the embodiments of the present invention used for the anticancer treatment of the breast, liver, blood and lung cancer respectively.

In the first embodiment, MDA-MB-231, a human breast cancer cell and 4T1, a mouse breast cancer cell, Huh-7 and HepG2, a human liver cancer cell and BNL-1-MEA, a mouse liver cancer cell, HL60, a human blood cancer cell and WEHI-3, a mouse blood cancer cell , and

A549, a human lung cancer cell and LLC1, a mouse lung cancer cell are used for the experiments. A 96-well plate is used for cell culture. In the test, cells are treated with 0, 25, 50, 100, 200, 400 μg/mL of D2 fraction for 24 hours, and then subjected to cell viability assay (or MTT assay). In cell viability assay, cells are washed with 1X PBS once and treated with 0.5 mg/mL of MTT (3-(4,5-Dimethylthiazol-2-yl)-2,5-diphenyltetrazolium bromide). After incubation at 37 degrees Centigrade for 1-2 hours MTT is removed and cells are washed with 1X PBS once, followed by dimethyl sulfoxide (DMSO) to dissolve the cells. Finally optical density (OD) of 570 nm is detected and medium only is used as a 100% cell viability.

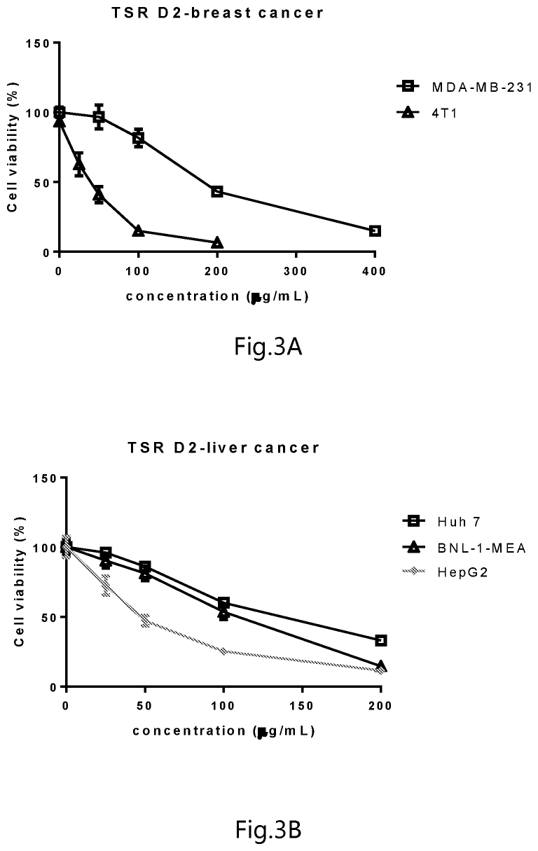

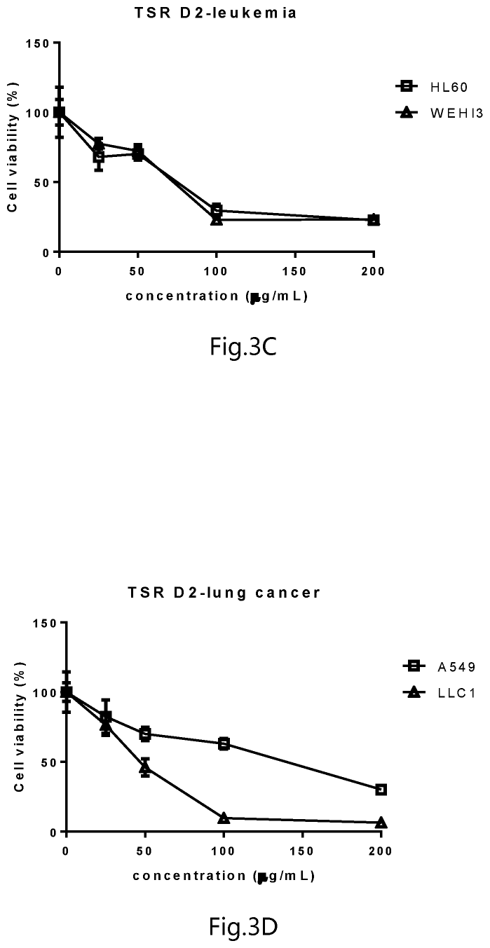

FIG. 3(A) shows the experimental results of MDA-MB-231, a human breast cancer cell and 4T1, a mouse breast cancer cell of an embodiment respectively, wherein the x-axis represents the concentration of D2 fraction and, the y-axis represents the percentage of cell viability. Two curves show the relation between D2 fraction concentration and the cell viability of the MDA-MB-231 human breast cancer cell indicated with square and that of 4T1 mouse breast cancer cell indicated with triangle. The results indicate that the treatment of D2 fraction can inhibit the growth of the breast cancer cells significantly when compared with the group without D2 fraction. Moreover the inhibition capability positively correlates to the concentration of the D2 fraction. The 50% inhibition concentrations of D2 fraction in 4T1 mouse breast cancer cell and in MDA-MB-231 human breast cancer cell are 38.2 μg/ml and 181.9 μg/ml respectively. Taken together, D2 fraction has shown a great anticancer effect on breast cancer cell.

FIG. 3 (B) shows the experimental results of Huh-7, a human liver cancer cell , HepG2, a human liver cancer cell , and BNL-1-MEA, a mouse liver cancer cell of an embodiment respectively, wherein the x-axis represents the concentration of D2 fraction and, the y-axis represents the percentage of cell viability. Three curves show the relation between D2 fraction concentration and the cell viability of the Huh-7 human liver cancer cell indicated in square, that of HepG2 human liver cancer cell indicated in diamond, and that of BNL-1-MEA mouse liver cancer cell indicated in triangle. The results indicate that the treatment of D2 fraction can inhibit the growth of liver cancer cells significantly when compared with the group without D2 fraction. Moreover, the inhibition capability positively correlates to the concentration of D2 fraction, and the 50% inhibition concentrations of D2 fraction in Huh-7 human liver cancer cell, in HepG2 human liver cancer cell and in BNL-1-MEA mouse liver cancer cell are 120.1 μg/ml, 48.6 μg/ml, and 106.9 μg/ml respectively. Above results show a great anticancer effect of D2 fraction in liver cancer cell.

FIG. 3(C) shows the experimental results of HL60, a human blood cancer cell and WEHI-3, a mouse blood cancer cell of an embodiment respectively, wherein the x-axis represents the concentration of D2 fraction and, the y-axis represents the percentage of cell viability. Two curves show the relation between D2 fraction concentration and the cell viability of the HL60 human blood cancer cell indicated with square and that of the WEHI-3 mouse blood cancer cell indicated with triangle. The results show that the treatment of D2 fraction can inhibit the growth of blood cancer cells significantly when compared with the group without D2 fraction. Moreover the inhibition capability positively correlates to the concentration of D2 fraction. The 50% inhibition concentrations of D2 fraction in HL60 human blood cancer cell and in WEHI-3 mouse blood cancer cell are 65.7 μg/ml and 66.4 μg/ml respectively. Taken together, D2 fraction has shown a great anticancer effect on blood cancer cell.

FIG. 3(D) shows the experimental results of A549, a human lung cancer cell and LLC1, a mouse lung cancer cell of an embodiment respectively, wherein the x-axis represents the concentration of D2 fraction and, the y-axis represents the percentage of cell viability. Two curves show the relation between D2 fraction concentration and the cell viability of A549 human lung cancer cell indicated with square and LLC1 mouse lung cancer cell indicated with triangle. The results indicate that the treatment of D2 fraction can inhibit the growth of lung cancer cells when compared with the group without D2 fraction. Moreover, the inhibitory capability is directly proportional to the concentration of D2 fraction. The 50% inhibition concentrations of D2 fraction in A549 human lung cancer cell and in LLC1 mouse lung cancer cell are 124.5 μg/ml and 46.2 μg/ml respectively. Taken together, D2 fraction has shown a great anticancer effect on lung cancer cell.

The results of above embodiment show that treatment of D2 fraction for 24 hours can inhibit the growth of MDA-MB-231 and 4T1 breast cancer cell, Huh-7, HepG2, and BNL-1-MEA liver cancer cell, HL60 and WEHI-3 blood cancer cell, and A549 and LLC1 lung cancer cell significantly. The 50% inhibition concentration of D2 fraction in different cells ranges from 38.2 to 181.9 μg/ml. Therefore, D2 fraction can be developed as anticancer medicine.

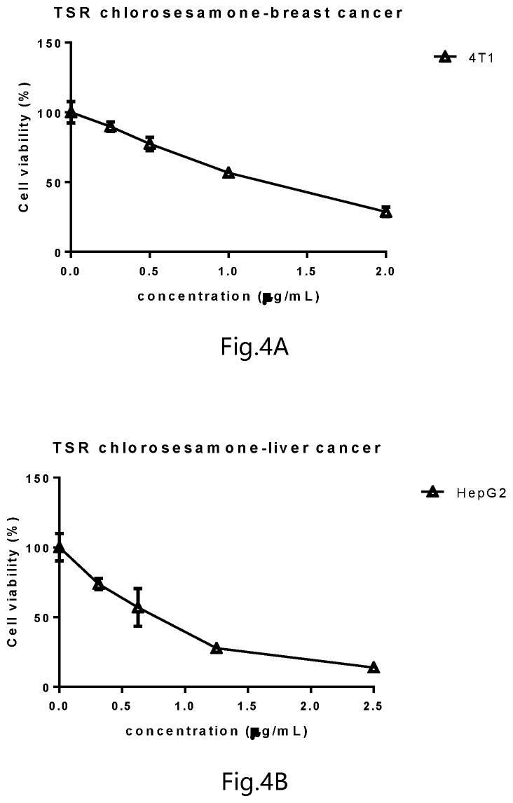

FIG. 4(A) shows the experimental result of 4T1 mouse breast cancer cell of an embodiment, wherein the x-axis represents the concentration of the chlorosesamone, and the y-axis represents the percentage of cell viability. The results indicate that the treatment of chlorosesamone can inhibit the growth of cells dramatically when compared with the group without chlorosesamone. Moreover, the inhibition capability positively correlates to the concentration of chlorosesamone. The 50% inhibition concentration of chlorosesamone in 4T1 mouse breast cancer cell is 1.3 μg/ml. Obviously, chlorosesamone has shown a significant anticancer effect on breast cancer cell.

FIG. 4(B) shows the experimental results of HepG2 human liver cancer cell of an embodiment, wherein the x-axis represents the concentration of the chlorosesamone, and the y-axis represents the percentage of cell viability. The results indicate that the treatment of chlorosesamone can inhibit the growth of cells dramatically when compared with the group without chlorosesamone. Moreover, the inhibition capability positively correlates to the concentration of chlorosesamone. The 50% inhibition concentration of chlorosesamone in HepG2 human liver cancer cell is 0.8 μg/ml. Obviously, chlorosesamone has shown a significant anticancer effect on liver cancer cell.

The results of above embodiment show that the chlorosesamone can inhibit the growth of 4T1 mouse breast cancer cell and HepG2 human liver cancer cell dramatically. This 50% inhibition concentration of chlorosesamone ranges from 0.8 to 1.3 μg/ml. Taken together, chlorosesamone is the major anticancer compound in D2 fraction.

What is claimed is:

1. A use of a D2 fraction obtained from the root of a sesame plant, being used to prepare a medicine for treating cancers , wherein the manufacturing method of the D2 fraction includes the following steps:
an extraction step to extract roots of the sesame plants at room temperature to obtain a crude extract; and
a separation step to separate an ethyl acetate layer from crude extract of extraction step to obtain fractions of D1 and D2; wherein D2 fraction contains chlorosesamone.

2. The method as claimed in claim 1, wherein the extraction step is performed by organic solvent or a supercritical extraction method.

3. The method as claimed in claim 2, wherein the separation step further uses a Diaion HP20 column, which is sequentially eluted by H₂O/MeOH (weight ratio 40/60), 100% MeOH, 100% acetone, H2O/acetone (weight ratio 30/70), for the separation.

4. A use of chlorosesamone, being used to prepare a medicine for treating cancers.

5. The use as claimed in claim 4, wherein the Chlorosesamone is extracted from the roots of the sesame plant.
